(19) United States
(12) Patent Application Publication
(10) Pub. No.: US 2026/0151217 A1
(43) Pub. Date: Jun. 4, 2026

(54) USING GAUGE PRESSURE FOR INFLATING AN IMPLANTABLE MEDICAL DEVICE

(71) Applicant: Boston Scientific Scimed, Inc., Maple Grove, MN (US)

(72) Inventors: Natalie Ann Borgos, Roseville, MN (US); Hui Li, Arden Hills, MN (US); Sean Thomas Horan, Minneapolis, MN (US); Jessica Williams, Marlborough, MA (US); John D. Hatlestad, Maplewood, MN (US)

(21) Appl. No.: 19/408,097

(22) Filed: Dec. 3, 2025

Related U.S. Application Data
(60) Provisional application No. 63/727,974, filed on Dec. 4, 2024.

(51) Int. Cl. A61F 2/00; A61F 5/41

(52) U.S. Cl. CPC A61F 2/004; A61F 5/41; A61F 2005/415; A61F 2250/0002; A61F 2250/0012; A61F 2250/0013

(57) ABSTRACT

According to an aspect, an implantable medical device includes an electronic pump device configured to transfer fluid between a fluid reservoir and an inflatable member to inflate or deflate the inflatable member, and an external controller configured to wirelessly communicate with the electronic pump device. The external controller or the electronic pump device is configured to receive a selected rigidity level from a user, convert the selected rigidity level to a target pressure value, obtain an ambient pressure value, compute a gauge pressure value based on the target pressure value and the ambient pressure value, and control inflation or deflation of the inflatable member using the gauge pressure value.

### CROSS-REFERENCE TO RELATED APPLICATION

This application claims priority to U.S. Provisional Patent Application No. 63/727,974, filed on Dec. 4, 2024, entitled “USING GAUGE PRESSURE FOR INFLATING AN IMPLANTABLE MEDICAL DEVICE”, the disclosure of which is incorporated by reference herein in its entirety.

### TECHNICAL FIELD

This disclosure relates generally to an implantable medical device for computing gauge pressure of an inflatable member using a selected rigidity level and using the gauge pressure to control inflation and/or deflation of the inflatable member.

### BACKGROUND

Some implantable medical devices have an inflatable member and a pump device. The pump device may be manually operated to inflate or deflate the inflatable member. The use of an electronic pump device may present difficulties in measuring rigidity of the inflatable member.

### SUMMARY

In some aspects, the techniques described herein relate to an implantable medical device including: an electronic pump device configured to transfer fluid between a fluid reservoir and an inflatable member to inflate or deflate the inflatable member; and an external controller configured to wirelessly communicate with the electronic pump device, the external controller or the electronic pump device configured to: receive a selected rigidity level from a user; convert the selected rigidity level to a target pressure value; obtain an ambient pressure value; compute a gauge pressure value based on the target pressure value and the ambient pressure value; and control inflation or deflation of the inflatable member using the gauge pressure value.

In some aspects, the techniques described herein relate to a method including: receiving a selected rigidity level from a user; converting the selected rigidity level to a target pressure value; obtaining an ambient pressure value; computing a gauge pressure value based on the target pressure value and the ambient pressure value; and controlling inflation or deflation of an inflatable member using the gauge pressure value.

In some aspects, the techniques described herein relate to an implantable medical device including: an electronic pump device configured to transfer fluid between a fluid reservoir and an inflatable member to inflate or deflate the inflatable member; and an external controller configured to wirelessly communicate with the electronic pump device, the external controller or the electronic pump device configured to: receive a selected rigidity level from a user; convert the selected rigidity level to a target pressure value; obtain an ambient pressure value; compute a gauge pressure value based on the target pressure value and the ambient pressure value; and control inflation or deflation of the inflatable member using the gauge pressure value.

In some aspects, the techniques described herein relate to an implantable medical device including: at least one processor; and at least one non-transitory computer-readable medium storing executable instructions that when executed by the at least one processor execute operations, the operations including: receiving a selected rigidity level from a user; converting the selected rigidity level to a target pressure value; obtaining an ambient pressure value; computing a gauge pressure value based on the target pressure value and the ambient pressure value; and controlling inflation or deflation of an inflatable member using the gauge pressure value.

In some aspects, the techniques described herein relate to a method including: receiving a selected rigidity level from a user; converting the selected rigidity level to a target pressure value; obtaining an ambient pressure value; computing a gauge pressure value based on the target pressure value and the ambient pressure value; and controlling inflation or deflation of an inflatable member using the gauge pressure value.

### BRIEF DESCRIPTION OF THE DRAWINGS

FIG. 1A illustrates an implantable medical device for computing a gauge pressure value from a selected rigidity level according to an aspect.

FIG. 1B illustrates an example of the implantable medical device according to another aspect.

FIG. 1C illustrates an example of a user interface with a rigidity level control according to an aspect.

FIG. 1D illustrates an ambient pressure calculation engine for computing an ambient pressure value according to an aspect.

FIG. 1E illustrates an ambient pressure calculation engine for computing an ambient pressure value according to another aspect.

FIG. 2 illustrates an example of an exploded view of an electronic pump device according to an aspect.

FIG. 3 illustrates a perspective of an inflatable penile prosthesis according to an aspect.

FIG. 4 illustrates an example of an artificial urinary sphincter device according to an aspect.

FIG. 5 illustrates a flowchart depicting example operations of computing the ambient pressure value according to an aspect.

### DETAILED DESCRIPTION

This disclosure relates generally to an implantable medical device for computing a gauge pressure value of an inflatable member from a selected rigidity level, and, using the gauge pressure value to control the inflation or deflation of the inflatable member. The gauge pressure computed by the implantable medical device may represent a level of hardness or rigidity of the inflatable member. The computed gauge pressure value is used by an electronic pump device to activate one or more pumps to inflate the inflatable member (e.g., transfer fluid from a fluid reservoir to the inflatable member) such that the inflatable member achieves the gauge pressure value.

The implantable medical device includes an inflatable member, a fluid reservoir, and an electronic pump device that transfers fluid between the fluid reservoir and the inflatable member. In some examples, the implantable medical device includes an inflatable penile prosthesis with one or more inflatable cylinders. In some examples, the implantable medical device includes a urinary control device with an inflatable cuff. The electronic pump device may automatically transfer fluid between the inflatable member and the fluid reservoir. The electronic pump device communicates with an external controller. A user may use the external controller to inflate and/or deflate the inflatable member, including selecting a desired rigidity level. In some examples, the external controller may be a client application executable by a user device.

The operations to compute gauge pressure may be performed by the electronic pump device, the external controller, or a combination of the electronic pump device and the external controller. The implantable medical device receives, from a user, a selected rigidity level about a target rigidity of the inflatable member. For example, the user may use the external controller to enter or select a desired rigidity level among a plurality of rigidity levels within a first scale (or range) (e.g., 0% to 100%). In some examples, a selected rigidity level is referred to a desired level of rigidity, and the first scale is a range of rigidity levels or values. The rigidity levels may be different levels within the first scale (e.g., 5%, 10%, 20% etc.). Although some examples use percentages as the first scale, the first scale may include other ranges such as a numerical range. In some examples, the rigidity levels correspond to different levels of hardness or rigidity of the inflatable member. In some examples, the external controller displays a user interface with one or more device controls to control the inflation and/or deflation of the inflatable member. The device control(s) includes a rigidity level control that allows the user to enter or select a desired rigidity level. In some examples, the rigidity level control includes a movable slider, where a position of the movable slider selects a particular rigidity level.

The implantable medical device converts the selected rigidity level to a target pressure value. The target pressure value may be a numerical value within a second scale. In some examples, the target pressure value is a value compatible with a pressure sensor used for generating pressure readings about the pressure of the inflatable member. In some examples, the target pressure value is a target measurement unit of pressure such as pounds per square inch (PSI), and the target pressure value is within a PSI range (e.g., 0 PSI to 16.5 PSI). In some examples, the implantable medical device converts the rigidity level expressed in terms of a percentage to a target pressure value expressed in terms of PSI. In some examples, the implantable medical device uses a function, which may be a linear function, non-linear function, or a statistical method.

The implantable medical device obtains an ambient pressure value about the ambient pressure of the implantable medical device. In some examples, the ambient pressure value is referred to as atmospheric pressure. In some examples, the ambient pressure value is referred to as a local atmospheric pressure. In some examples, the external controller or the electronic pump device may obtain the ambient pressure value from a memory device.

In some examples, the implantable medical device computes (e.g., periodically computes) the ambient pressure value and stores the updated ambient pressure value in the memory device. In some examples, the electronic pump device or the external controller computes the ambient pressure value. In some examples, the external controller includes an environmental pressure sensor configured to measure ambient pressure, and the external controller stores the ambient pressure value in its memory device, which can also be transmitted and stored on a memory device on the electronic pump device. In some examples, the electronic pump device includes a pressure sensor connected to the fluid reservoir, and the pressure readings about the pressure of the fluid reservoir are used to compute the ambient pressure value. For example, the pressure readings about the pressure of the fluid reservoir may be processed to provide a measure of ambient pressure. In some examples, the electronic pump device includes an environmental pressure sensor configured to measure the ambient pressure.

The implantable medical device computes a gauge pressure value based on the target pressure value and the ambient pressure value. The target pressure value and the ambient pressure value may be different pressure values on the same scale (e.g., the second scale). The implantable medical device may compute the gauge pressure value by offsetting the target pressure value with the ambient pressure value (e.g., P_{gauge}=[P_{target}−P_{ambient}]).

In some examples, the implantable medical device may obtain a correction value from a memory device, and the implantable medical device computes the gauge pressure value by offsetting the target pressure value with the ambient pressure value and the correction value (e.g., P_{gauge}=[P_{target}−P_{ambient}+/−P_{correction}). The correction value may be a positive value or a negative value in the second scale (e.g., the pressure scale). The correction value may be a correction term to account for pressure measurement error during pumping (e.g., inflation) and/or to account for pressure drop post-inflation due to cylinder relaxation. In some examples, the correction value may be specific to a type of the inflation member (e.g., cylinder-specific) and/or patient data. In some examples, the implantable medical device may update the correction value, over time, to account for material relaxation. In some examples, the implantable medical device receives one or more metrics about the usage of the implantable medical device (e.g., inflation time, deflation time, pressure readings, etc.) and can update the correction value based on the metrics(s).

The implantable medical device may control inflation of the inflatable member using the gauge pressure value. For example, the electronic pump device includes a pressure sensor connected to the inflatable member, and the pressure sensor is configured to generate pressure readings about the pressure of the inflatable member. In response to an inflate signal, the electronic pump device activates one or more pumps to automatically transfer fluid from the fluid reservoir to the inflatable member such that the inflatable member achieves the pressure value provided by the gauge pressure value. In some examples, the electronic pump device receives the gauge pressure value from the external controller. For example, the external controller may compute the gauge pressure value and transmit the gauge pressure value to the electronic pump device to initiate inflation of the inflatable member. In some examples, the electronic pump device receives the selected rigidity level (e.g., expressed in terms of a percentage) from the external controller, the electronic pump device computes the gauge pressure value and uses the gauge pressure value to control inflation of the inflatable member. In some examples, the operations of computing a gauge pressure value are the same with respect to deflating the inflatable member to the selected rigidity level.

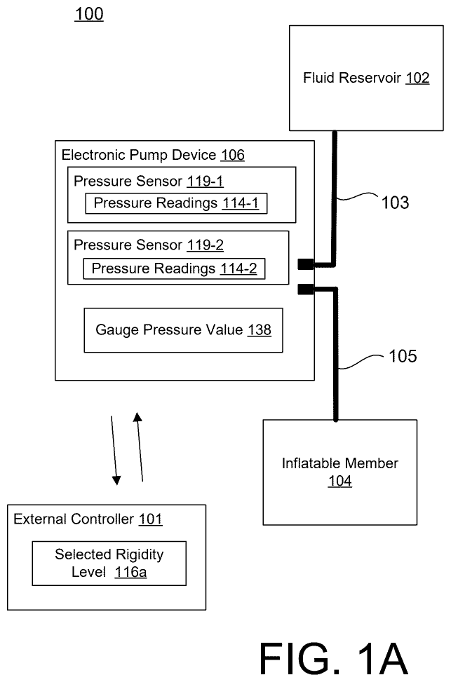

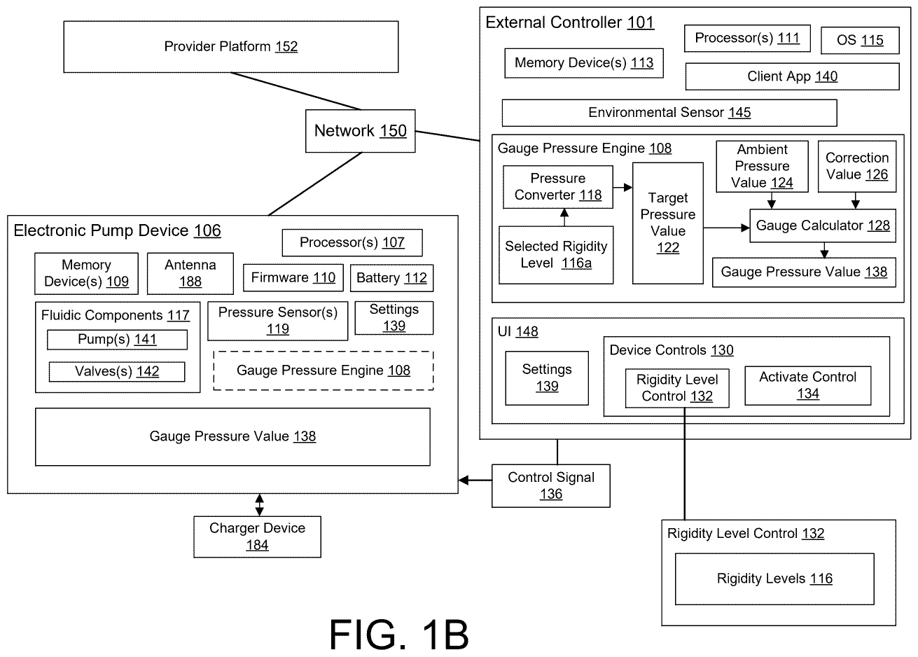

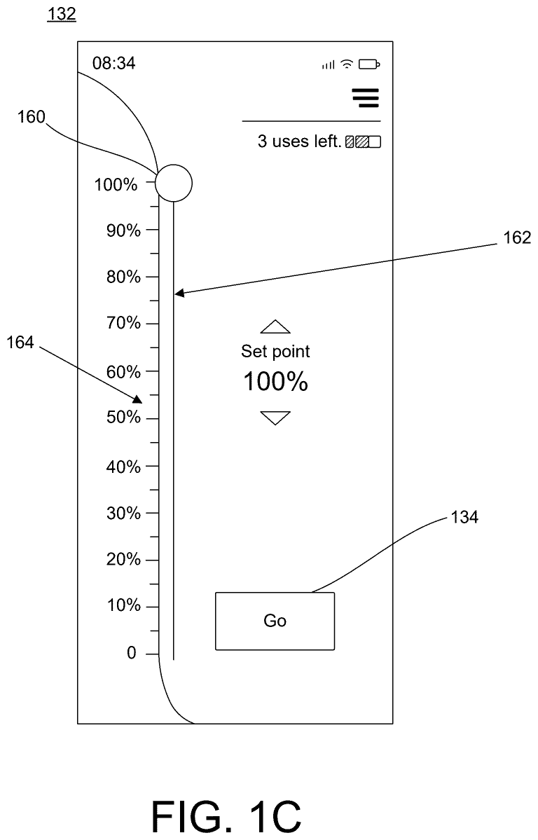

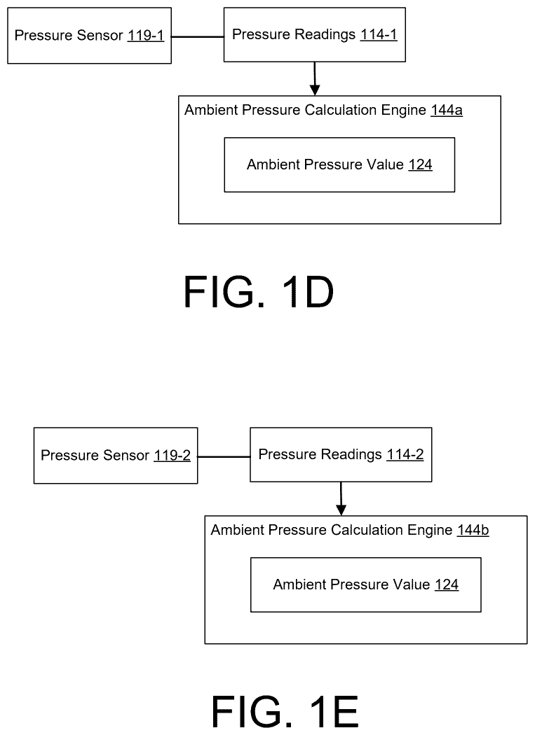

FIGS. 1A to 1E illustrate an implantable medical device 100 that computes a gauge pressure value 138 from a selected rigidity level 116a and uses the gauge pressure value 138 to control inflation or deflation of an inflatable member 104. The gauge pressure value 138 is an indication of user-desired rigidity for the inflatable member 104. In other words, the gauge pressure value 138 computed by the implantable medical device 100 may represent a desired level of hardness or rigidity of the inflatable member 104. Users want to inflate the inflatable member 104 to a desired rigidity, but there are one or more technical challenges for measuring rigidity (e.g., directly measuring rigidity) of the inflatable member 104 while the inflatable member 104 is in the body of a patient. However, the implantable medical device 100 uses the techniques herein to compute a gauge pressure value 138 as a representation (or substitute) of rigidity from a user-selected rigidity level 116a and uses the gauge pressure value 138 to inflate or deflate the inflatable member 104 to the desired rigidity. In some examples, the implantable medical device 100 is an artificial urinary sphincter device. In some examples, the implantable medical device 100 is an inflatable penile prosthesis. However, the implantable medical device 100 may include any type of medical device that transfers fluid between components of the implantable medical device 100.

As shown in FIG. 1A, the implantable medical device 100 includes a fluid reservoir 102, an inflatable member 104, and an electronic pump device 106 configured to transfer fluid between the fluid reservoir 102 and the inflatable member 104. In some examples, the inflatable member 104 is an inflatable cuff member configured to be implemented around a urethra of a patient. In some examples, the inflatable member 104 is a penile prosthetic with one or more inflatable cylinders that may be implanted into the corpus cavernosum of the user. The fluid reservoir 102 may be implanted in the abdomen or pelvic cavity of the user (e.g., the fluid reservoir 102 may be implanted in the lower portion of the user's abdominal cavity or the upper portion of the user's pelvic cavity). In some examples, at least a portion of the electronic pump device 106 may be implemented in the patient's body.

The inflatable member 104 may be capable of expanding upon the injection of fluid into a cavity of the inflatable member 104. If implanted around the urethra, the expansion of the inflatable member 104 causes the urethra to become restricted, thereby reducing the risk of incontinence in patients. For example, the electronic pump device 106 is configured to move fluid to pressure the inflatable cuff (e.g., the inflatable member 104), which constricts the urethra, thereby restricting the flow of urine. To urinate, the patient may operate the electronic pump device 106 to depressurize the inflatable cuff by transferring fluid from the inflatable cuff to the fluid reservoir 102. If implanted into the corpus cavernosum, upon injection of the fluid into the inflatable member 104, the inflatable member 104 may increase its length and/or width, as well as increase its rigidity.

The fluid reservoir 102 may include a container having an internal chamber configured to hold or house fluid that is used to inflate the inflatable member 104. In some examples, the fluid reservoir 102 is pressurized. In some examples, the fluid reservoir 102 is a pressurized balloon. In some examples, the implantable medical device 100 includes a single pressurized balloon. In some examples, the implantable medical device 100 includes two or more pressurized balloons. The pressure in the inflatable member 104 may be generated by the fluid reservoir 102. In some examples, the pressure in the fluid reservoir 102 is greater than the pressure in the inflatable member 104 (e.g., even when the inflatable member 104 is at its target or maximum pressure). In some examples, the pressure in the fluid reservoir 102 is always greater than the pressure in the inflatable member 104.

The implantable medical device 100 may include a first tube member 103 and a second tube member 105. In some examples, the first tube member 103 and the second tube member 105 are referred to as conduit connectors. Each of the first tube member 103 and the second tube member 105 may define a lumen configured to transfer the fluid to and from the electronic pump device 106. The first tube member 103 may be coupled to the electronic pump device 106 and the fluid reservoir 102 such that fluid can be transferred between the electronic pump device 106 and the fluid reservoir 102 via the first tube member 103. For example, the first tube member 103 may define a first lumen configured to transfer fluid between the electronic pump device 106 and the fluid reservoir 102. The first tube member 103 may include a single or multiple tube members for transferring the fluid between the electronic pump device 106 and the fluid reservoir 102. In some examples, the first tube member 103 may be referred to as first tube members, and two first tube members can be connected together using a connector.

The second tube member 105 may be coupled to the electronic pump device 106 and the inflatable member 104 such that fluid can be transferred between the electronic pump device 106 and the inflatable member 104 via the second tube member 105. For example, the second tube member 105 may define a second lumen configured to transfer fluid between the electronic pump device 106 and the inflatable member 104. The second tube member 105 may include a single or multiple tube members for transferring the fluid between the electronic pump device 106 and the inflatable member 104. In some examples, the second tube member 105 may be referred to as second tube members, and two second tube members can be connected together using a connector. In some examples, the first tube member 103 and the second tube member 105 may include a silicone rubber material. In some examples, the electronic pump device 106 may be directly connected to the fluid reservoir 102.

The electronic pump device 106 may automatically transfer fluid between the fluid reservoir 102 and the inflatable member 104 without the user manually operating a pump (e.g., squeezing and releasing a pump bulb). In some examples, the electronic pump device 106 is referred to as a can. The electronic pump device 106 includes one or more processors 107 and one or more memory devices 109. The memory device(s) 109 may store executable instructions that when executed by the processor(s) 107 to execute operations discussed herein with respect to the electronic pump device 106. The memory device(s) 109 may store firmware 110. The electronic pump device 106 includes a battery 112, configured to be charged to be by a charger device 184. In some examples, the firmware 110 may be an operating system of the electronic pump device 106. In some examples, the memory device(s) 109 include a non-transitory computer-readable medium or computer program product.

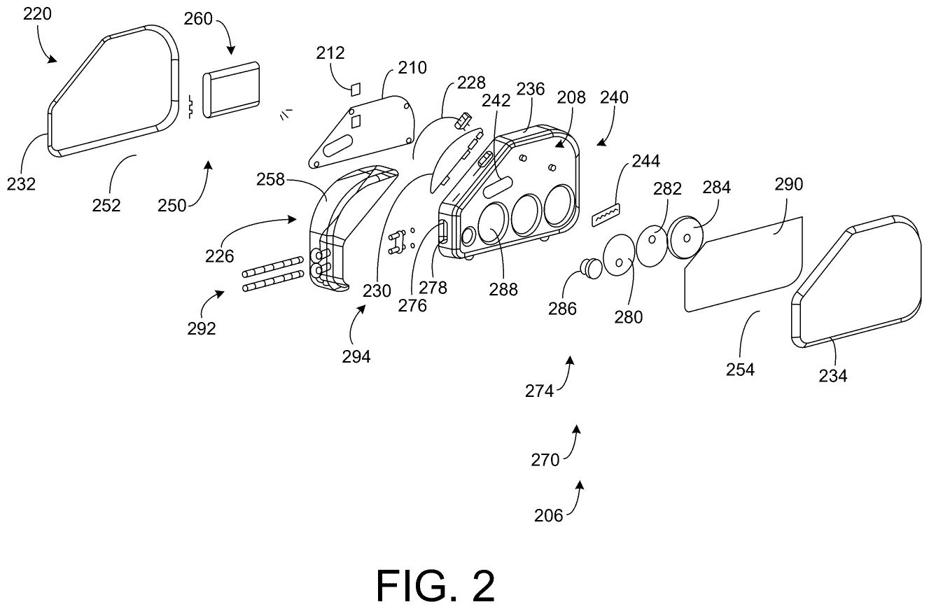

The electronic pump device 106 includes one or more pressure sensors 119. In some examples, the electronic pump device 106 includes a single pressure sensor 119. In some examples, the electronic pump device 106 includes two or more pressure sensors 119. The electronic pump device 106 that can monitor, control, and/or regulate the pressure within an inflatable member 104 using the pressure sensor(s) 119. As shown in FIG. 1A, the electronic pump device 106 may include a pressure sensor 119-1 connected to the inflatable member 104. The pressure sensor 119-1 is used for measuring the pressure (e.g., inflation pressure or cylinder pressure) of the inflatable member 104. In some examples, the pressure sensor 119-1 is coupled to a fluid port (e.g., first port 276 or second port 278 of FIG. 2) inside of a housing of the electronic pump device 106, where the fluid port is in fluid communication with the inflatable member 104. In some examples, the pressure sensor 119-1 is aligned (e.g., in-line) with the fluid port fluidly connected to the inflatable member 104. In some examples, the pressure sensor 119-1 is coupled to a circuit substrate (e.g., the circuit substrate 210 of FIG. 2). The pressure sensor 119-1 may generate pressure readings 114-1 according to a sampling rate. Each pressure reading 114-1 includes a pressure value, and, in some examples, a timestamp.

As shown in FIG. 1A, the electronic pump device 106 may include a pressure sensor 119-2 connected to the fluid reservoir 102. In some examples, the pressure sensor 119-2 is coupled to a fluid port (e.g., first port 276 or second port 278 of FIG. 2) inside of a housing of the electronic pump device 106, where the fluid port is in fluid communication with the fluid reservoir 102. In some examples, the pressure sensor 119-2 is aligned (e.g., in-line) with the fluid port. In some examples, the pressure sensor 119-2 is coupled to a circuit substrate (e.g., the circuit substrate 210 of FIG. 2). The pressure sensor 119-2 is used for measuring a pressure (e.g., reservoir pressure) of the fluid reservoir 102.

The electronic pump device 106 includes fluidic components 117 configured to enable the transfer of fluid between the inflatable member 104 and the fluid reservoir 102. The fluidic components 117 may include one or more pumps 141 (e.g., electronic pumps, e.g., pumps operated by electrical signals) and one or more fluid valves 142. The electronic pump device 106 includes an antenna 188 configured to wirelessly transmit (and receive) wireless signals from an external controller 101. The external controller 101 may be any type of component that can communicate with the electronic pump device 106. The external controller 101 may be a computer, smartphone, tablet, pendant, wearable device (e.g., smartwatch, wristband, etc.), key fob, etc. A user may use the external controller 101 to control the inflation and deflation of the inflatable member 104.

In some examples, the external controller 101 may be any type of device having one or more processors 111, one or more memory devices 113, and an operating system 115. The memory device(s) 113 may store executable instructions that cause the external controller 101 (e.g., the client application 140) to execute operations discussed herein. In some examples, the memory device(s) 113 include a non-transitory computer-readable medium or computer program product.

In some examples, the external controller 101 includes a client application 140 executable by a user device such as a smartphone, wearable device, or generally any type of computing device. In some examples, the client application 140 is a patient application. In some examples, the client application 140 is a clinician application. The client application 140 is configured to enable a patient or clinician to control inflation and deflation of the inflatable member 104 and to set or configure settings 139 associated with the implantable medical device 100. In some examples, the client application 140 is installed on an operating system 115 of the external controller 101. In some examples, the client application 140 is the operating system 115 or a sub-component of the operating system 115. In some examples, the client application 140 is a mobile (native) application. In some examples, the client application 140 is a web application executable by a browser application. In some examples, the client application 140 is a website of the provider platform 152.

In some examples, the electronic pump device 106 communicates with the external controller 101, over a network 150, which may be a Wi-Fi connection, a mobile network connection, or a short-range communication network such as Bluetooth. In some examples, the electronic pump device 106 can communicate directly with a provider platform executable by one or more server computers. The server computer(s) includes one or more processors and one or more memory devices. In some examples, the electronic pump device 106 and the external controller 101 communicate with each other via a short-range communication network, and the external controller 101 and the provider platform 152 communicate with each other via a Wi-Fi or mobile network communication network.

The client application 140 is configured to display a user interface 148 on a display of the external controller 101. In some examples, the client application 140 is associated with a user account. In some examples, the user account may store information about the patient and/or the implantable medical device 100 such as patient data and/or device data. In some examples, the user account may include settings 139 of the implantable medical device 100. In some examples, the user account may include collected data such as charging data and/or usage data. In some examples, information (or a portion thereof) of the user account may be stored at the provider platform 152 and/or the electronic pump device 106 and/or locally on the external controller 101. The charging data may be information about the user's charging activity, such as the frequency of charging, the charging levels, etc. In some examples, the usage data may include information about the use of the implantable medical device 100 such as the frequency of inflate and/or deflate states, the duration of inflate and/or deflate states, and/or the pressure levels during the inflate or deflate states (e.g., including gauge pressure values 138 set by the user).

The user interface 148 includes an interface for defining, adjusting, or setting one or more settings 139. The user interface 148 may include device controls 130 for controlling the inflation and deflation of the inflatable member 104. In some examples, the device controls 130 are UI elements. The device controls 130 includes a rigidity level control 132 configured to enable the user to select a rigidity level 116a to set the rigidity of the inflatable member 104. In some examples, the device controls 130 include an activate control 134, which, when selected, causes the external controller 101 to generate and send a control signal 136 with the gauge pressure value 138 or the selected rigidity level 116a.

The external controller 101 (e.g., the client application 140) receives, from a user, a selected rigidity level 116a about a target rigidity of the inflatable member 104. For example, the user may use the external controller 101 to enter or select a desired rigidity level 116 a among a plurality of rigidity levels 116 within a first scale (or range) (e.g., 0% to 100%). In some examples, a selected rigidity level 116 a is referred to a desired level of rigidity, and the first scale is a range of rigidity levels or values. The rigidity levels 116 may be different levels within the first scale (e.g., 5%, 10%, 20% etc.). Although some examples use percentages as the first scale, the first scale may include other ranges such as a numerical range. In some examples, the rigidity levels 116 correspond to different levels of hardness or rigidity of the inflatable member 104.

In some examples, the external controller 101 displays the user interface 148 with the rigidity level control 132 to enable the user to select or enter a selected rigidity level 116a. In some examples, the rigidity level control 132 includes a slider. As shown in FIG. 1C, the rigidity level control 132 includes a track 162, and a user interface element 160, movable by the user, along the track 162. The rigidity level control 132 including indicators 164 representing the plurality of rigidity levels 116. A position of the user interface element 160 in the track 162 indicates the selected rigidity level 116.

In some examples, the external controller 101 includes a gauge pressure engine 108. The gauge pressure engine 108 is configured to receive the selected rigidity level 116a and to compute the gauge pressure value 138. The gauge pressure engine 108 includes a pressure converter 118 that converts the selected rigidity level 116a to a target pressure value 122. The target pressure value 122 may be a numerical value within a second scale. In some examples, the target pressure value 122 is a value compatible with the pressure sensor 119-1 used for generating pressure readings 114-1 about the pressure of the inflatable member 104.

In some examples, the target pressure value 122 is a target measurement unit of pressure such as pounds per square inch (PSI), and the target pressure value 122 is within a PSI range (e.g., 0 PSI to 16.5 PSI). In some examples, the pressure converter 118 converts the selected rigidity level 116a expressed in terms of a percentage to a target pressure value 122 expressed in terms of PSI. In some examples, the pressure converter 118 uses a function, which may be a linear function, non-linear function, or a statistical method.

The gauge pressure engine 108 obtains an ambient pressure value 124 about the ambient pressure of the implantable medical device 100. The ambient pressure value 124 may be the pressure of the tissues and/or fluids that surround the implantable medical device 100, which may be influenced by body position, fluid levels, and/or muscle activity. In some examples, the ambient pressure value 124 may be dependent upon altitude, temperature, humidity, and/or other weather conditions. In some examples, the ambient pressure value 124 is referred to as atmospheric pressure. In some examples, the ambient pressure value 124 is referred to as a local atmospheric pressure.

In some examples, the gauge pressure engine 108 obtains the ambient pressure value 124 from a memory device 113 of the external controller 101. In some examples, the gauge pressure engine 108 obtains the ambient pressure value 124 from an environmental sensor 145 on the external controller 101. The environmental sensor 145 may directly measure the ambient pressure value 124. In some examples, the gauge pressure engine 108 obtains the ambient pressure value 124 from the operating system 115 of the external controller 101.

In some examples, the gauge pressure engine 108 computes the ambient pressure value 124 from the pressure readings 114-1 or the pressure readings 114-2, or a combination of the pressure readings 114-1 and the pressure readings 114-2 and stores the ambient pressure value 124 in the memory device 113. In some examples, the external controller 101 may transmit the ambient pressure value 124 to the electronic pump device 106, and the electronic pump device 106 stores the ambient pressure value 124 at the memory device 109 of the electronic pump device 106. The electronic pump device 106 may use the ambient pressure value 124 to offset the pressure value from the pressure reading 114-1 to determine whether the pressure of the inflatable member 104 achieves the gauge pressure value 138. In some examples, the electronic pump device 106 computes the ambient pressure value 124 from the pressure readings 114-1 or the pressure readings 114-2, or a combination of the pressure readings 114-1 and the pressure readings 114-2 and stores the ambient pressure value 124 in the memory device 109. In some examples, the electronic pump device 106 transmits the ambient pressure value 124 to the external controller 101 for storage in the memory device 113.

In some examples, as shown in FIG. 1D, the implantable medical device 100 includes an ambient pressure calculation engine 144a configured to compute the ambient pressure value 124 from the pressure readings 114-1 of the pressure sensor 119-1, e.g., the sensor connected to the inflatable member 104. The ambient pressure calculation engine 144a may be stored on the electronic pump device 106 or the external controller 101.

In some examples, as shown in FIG. 1E, the implantable medical device 100 includes an ambient pressure calculation engine 144b configured to compute the ambient pressure value 124 from the pressure readings 114-2 of the pressure sensor 119-2, e.g., the sensor connected to the fluid reservoir 102. In some examples, bias on the reservoir sensor (e.g., the pressure sensor 119-2) may be caused by IAP changes and/or external pressures, which typically increases pressure (e.g., only increases). By using a pressure sensor 119-2 connected to the fluid reservoir 102 for computing the ambient pressure value 124, the implantable medical device 100 may be less susceptible to being affected (e.g., significantly affected) by temporary or minor errors or inconsistencies in measurements (e.g., it can handle short-term fluctuations or inaccuracies in data without being significantly impacted).

The ambient pressure calculation engine 144b may activate the pressure sensor 119-2 to generate a signal with pressure readings 114-2 during a time interval. The time interval may have a predetermined length (e.g., thirty seconds, one minute, two minutes, or three minutes, or generally any set length of time). In some examples, a time interval is referred to as a sampling period or a measurement period. In some examples, the ambient pressure calculation engine 144b may periodically activate the pressure sensor 119-2 to generate the signal for a respective sampling period and determine whether to update the ambient pressure value 124. In some examples, updating the ambient pressure value 124 includes replacing an old value with a new value in a memory device.

The ambient pressure calculation engine 144b activates the pressure sensor 119-2 to generate the signal with the pressure readings 114-2 during the time interval in response to the detection of a triggering event. In some examples, the triggering event may be expiration of a timer or achieving a time indicated by a sampling schedule (e.g., one or more times a day, one or more times a week, etc.). The pressure sensor 119-2 may generate the signal with pressure readings 114-2 according to a sampling rate. In some examples, the sampling rate is between 1-10 Hz. Each pressure reading 114-2 includes a pressure value, and, in some examples, a timestamp.

The ambient pressure calculation engine 144b processes the pressure readings 114-2 to identify a portion of the pressure readings 114-2 that are within a certain percentile range of the pressure readings 114-2 during the time interval. The percentile range is defined by a percentile threshold (e.g., a low percentile threshold) and a percentile threshold (e.g., a high percentile threshold). In some examples, the percentile range includes or is less than the 50^{th} percentile of the pressure readings. In some examples, the percentile range is 5^{th} to 50^{th} percentile. In some examples, the percentile range is 5^{th} to 40^{th} percentile. In some examples, the percentile range is 5^{th} to 20^{th} percentile. In some examples, the ambient pressure calculation engine 144b uses a lower percentile range to filter out pressure readings 114-2 caused by short-term drops in pressure and bias due to raised IAP. In some examples, the ambient pressure calculation engine 144b ranks the pressure readings 114-2 from the signal by the magnitude of the pressure values and selects the pressure readings 114-2 within the specified percentile range.

The ambient pressure calculation engine 144b generates an ambient pressure value 124 based on the portion of the pressure readings 114-2 from the signal that is within the percentile range. In some examples, the ambient pressure calculation engine 144b selects a pressure value from one of the pressure readings 114-2 from the signal that is within the percentile range. In some examples, the ambient pressure calculation engine 144b selects a pressure reading 114-2 with a minimum pressure value among the portion of the pressure readings 114-2 that are within the percentile range. In some examples, the ambient pressure calculation engine 144b computes an average pressure value from the portion of the pressure readings 114-2 that are within the percentile range.

In some examples, the ambient pressure calculation engine 144b includes logic for determining whether to accept or reject the ambient pressure value 124 computed by the ambient pressure calculation engine 144b for a current time interval. For example, the ambient pressure calculation engine 144b may compute a threshold deviation using the portion of the pressure readings 114-2 (e.g., the pressure readings that fall within the percentile range).

In some examples, the threshold deviation is an absolute deviation. In some examples, the threshold deviation is a standard deviation. In some examples, the threshold deviation is a variance (e.g., averaged squared deviation from the mean). In some examples, the threshold deviation is peak width.

The ambient pressure calculation engine 144b may determine whether a deviation of the ambient pressure value 124 from the median is equal to or greater than the threshold deviation. In response to the deviation of the ambient pressure value 124 from the median being equal to or greater than the threshold deviation, the ambient pressure calculation engine 144b may discard (e.g., reject) the ambient pressure value 124. If rejected, the ambient pressure calculation engine 144b may wait until the next sampling period (e.g., in response to detection of a subsequent triggering event) to re-collect pressure readings to re-compute the ambient pressure value. In response to the deviation of the ambient pressure value 124 from the median being less than the threshold deviation, the ambient pressure calculation engine 144b may accept the ambient pressure value 124.

In some examples, when the ambient pressure value 124 is accepted, the ambient pressure calculation engine 144b may compare the ambient pressure value 124 for the current time interval with an ambient pressure value 124 from a previous time interval to determine whether to update the ambient pressure with the ambient pressure value 124 (e.g., a new ambient pressure value) for the current time interval or continue to maintain the previous ambient pressure value. In some examples, if the difference between the ambient pressure value 124 for the current time interval and the ambient pressure value 124 for the previous time interval is equal to or greater than a threshold level, the ambient pressure calculation engine 144b may update the ambient pressure with the ambient pressure value 124 for the current time interval.

If the difference between the ambient pressure value 124 for the current time interval and the ambient pressure value 124 for the previous time interval is less than the threshold level, the ambient pressure calculation engine 144b may use the previous ambient pressure value. In some examples, if the ambient pressure value 124 for the current time interval is less than the ambient pressure value 124 for the previous time interval, the ambient pressure calculation engine 144b uses the new ambient pressure value. In some examples, if the ambient pressure value 124 for the current time interval is greater than the previous ambient pressure value 124 by a threshold level, the ambient pressure calculation engine 144b uses the new ambient pressure value.

The gauge pressure engine 108 includes a gauge calculator 128 computes a gauge pressure value 138 based on the target pressure value 122 and the ambient pressure value 124. The target pressure value 122 and the ambient pressure value 124 may be different pressure values in the same scale (e.g., the second scale). The gauge calculator 128 may compute the gauge pressure value 138 by offsetting the target pressure value 122 with the ambient pressure value 124 (e.g., P_{gauge}=[P_{target}−P_{ambient}]).

In some examples, the gauge pressure engine 108 may obtain a correction value 126 from a memory device 113, and the gauge pressure engine 108 computes the gauge pressure value 138 by offsetting the target pressure value 122 with the ambient pressure value 124 and the correction value 126 (e.g., P_{gauge}=[P_{target}−P_{ambient}+/−P_{correction}). The correction value 126 may be a positive value or a negative value in the second scale (e.g., the pressure scale). The correction value 126 may be a correction term to account for pressure measurement error during pumping (e.g., inflation) and/or to account for pressure drop post-inflation due to cylinder relaxation. In some examples, the correction value 126 may be specific to a type of the inflation member (e.g., cylinder-specific) and/or patient data. In some examples, the gauge pressure engine 108 may update the correction value 126, over time, to account for material relaxation. In some examples, the gauge pressure engine 108 receives one or more metrics about the usage of the implantable medical device 100 (e.g., inflation time, deflation time, pressure readings, etc.) and can update the correction value 126 based on the metrics(s).

The external controller 101 may generate and transmit a control signal 136, where the control signal 136 includes the gauge pressure value 138. The electronic pump device 106 receives, via the antenna 188, the control signal 136, and the electronic pump device 106 inflates or deflates the inflatable member 104 such that the inflatable member achieves the gauge pressure engine 108.

In some examples, the electronic pump device 106 includes at least a portion of the gauge pressure engine 108. In some examples, the electronic pump device 106 may receive, over the network 150, a control signal 136 with the selected rigidity level 116a, and the electronic pump device 106 may generate the gauge pressure value 138 using the selected rigidity level 116a.

The electronic pump device 106 may control inflation of the inflatable member 104 using the gauge pressure value 138. For example, the pressure sensor 119-1 is configured to generate pressure readings 114-2 about the pressure of the inflatable member 104. In response to an inflate signal, the electronic pump device 106 activates one or more pumps 141 to automatically transfer fluid from the fluid reservoir 102 to the inflatable member 104 such that the inflatable member 104 achieves the pressure value provided by the gauge pressure value 138. The electronic pump device 106 may deactivate the pump(s) 141 in response to a pressure reading 114-1, from the pressure sensor 119-1, having a pressure value that achieves the gauge pressure value 138.

FIG. 2 illustrates an example of an exploded view of an electronic pump device 206 according to an aspect. The electronic pump device 206 may be an example of the electronic pump device 106 and may include any of the details discussed with reference to the other figures. The electronic pump device 206 includes a housing 220 with a first sidewall 232, a second sidewall 234, a peripheral wall 236, and a frame 240. The first sidewall 232, second sidewall 234, and the peripheral wall 236 are hermetically sealed together to form an internal compartment 250 within the housing 220.

The frame 240 is disposed within the internal compartment 250 to form a first partition 252 and a second partition 254 in such a manner that the first partition 252 is hermetically sealed from the second partition 254. The frame 240 can be integrally formed with the peripheral wall 236, the first sidewall 232, and/or the second sidewall 234. In some examples, the frame 240 is welded to the peripheral wall 236 or welded to the first sidewall 232, and/or the second sidewall 234. The first sidewall 232, the peripheral wall 236, and the frame 240 may form the first partition 252. The second sidewall 234, the peripheral wall 236, and the frame 240 may form the second partition 254, which is opposite the frame 240 from the first partition 252.

The electronic pump device 206 can include a header 226 attached to the housing 220 to form an internal region 258 between an inner surface of the header 226 and an outer surface of the housing 220 that includes power and communication interface structures such as a secondary coil 228 and the antenna 230 external to the hermetically sealed housing 220. The header 226 is configured from a dielectric or insulative material, such as a radome, to allow the transmission of power and communication signals between the antenna 230 and a handset programmer or charger, and between the secondary coil 228 and the charger. For example, the header 226 may include an over molded polymer affixed to the housing 220 and including the secondary coil 228 and the antenna 230 within the internal region 258. The secondary coil 228 and antenna 230 are constructed from a biocompatible material. In some examples, the secondary coil 228 and antenna 230 can be formed as a coil from a stamped titanium core clad with gold or silver. In some examples, the secondary coil 228 and antenna 230 can be formed from a gold wire.

The electronic pump device 206 includes an energy storage system, such as a battery (e.g., a rechargeable power source) (e.g., a rechargeable battery), and electronic components 212 within the first partition 252. The electronic components 212 can be disposed on a circuit substrate 210, such as a plurality of circuit boards, within the first partition 252. The battery 260 can assume various forms appropriate to provide power for generating desired electrical signals and to store power provided from the electronic components 212. For example, the battery 260 can incorporate lithium-ion (Li+) chemistry, e.g., a lithium-ion battery to operate the electronic components 212. In some examples, the electronic components 212 can be implemented by various components including resistors, capacitors, transistors, and integrated circuits disposed on the circuit substrate 210. The secondary coil 228 and antenna 230 are electrically coupled to the electronic components 212 within the first partition 252, such as via a hermetic feedthrough component.

The electronic components 212 can include a recharge system, a communication system, and a controller. The recharge system includes hardware configured to interface with the secondary coil 228 to receive power signals, and to provide the power signals in a form suitable to recharge the battery 260 and can include circuitry to reduce the likelihood of overcharging the battery 260. The communication system includes hardware configured to interface with the antenna 230 to receive electrical communication signals. For instance, the communication system can be configured to communicate via a wireless personal area network technology such as a short-range communication protocol (e.g., Bluetooth) (e.g., Bluetooth Low Energy), which is compatible with several operating systems that can be applied in mobile devices configured as external controllers (e.g., handset programmers). The communication system can include an integrated circuit to implement an applied communication technology. In some examples, the communication system can be used to transmit communication signals to other devices, such as a charger or the handheld programmer (e.g., external controller), and the communication system can be implemented to generate communication signals and provide the communication signals to the antenna 230 for transmission. In some examples, the communication system can be configured to receive and transmit radio frequency signals via the antenna 230. The controller can include a microcontroller to operate the recharge system and to receive and operate in response to communication signals or generate communication signals from the communication system.

The electronic pump device 206 also includes a fluidic circuit 270 within the second partition 254 and opposite the frame 240 from the battery 260 and electronic components 212. In some examples, the frame 240 can include an opening 242 that includes a hermetic interface 244, such as a feedthrough hermetically affixed to the frame 240. The electronic components 212 are operably coupled to the fluidic circuit 270 across the frame 240 via the hermetic interface 244. For example, the controller of the electronic components 212, powered by the battery 260, can cause the operation of the fluidic circuit 270 such as to control and monitor the fluidic circuit 270.

The fluidic circuit 270 includes a fluidic manifold 208 and fluidic components 274 operably coupled to the fluidic manifold 208. In some examples, the fluidic manifold 208 is a structure integrated into the frame 240 such that the fluidic manifold 208 and the frame 240 together form the hermetic barrier between the first partition 252 and the second partition 254 of the internal compartment 250. For instance, the battery 260, the circuit substrate 210, or electronic components 212 can be coupled to a first major surface of the fluidic manifold 208 in the first partition 252, and the fluidic components 274 are operably coupled to a second, and opposite major surface of the fluidic manifold 208 in the second partition 254.

The fluidic circuit 270 provides for the transfer of the fluid between the fluid reservoir (e.g., the fluid reservoir 102) and the inflatable member (e.g., the inflatable member 104). The fluidic manifold 208, which can be a hermetic manifold, segments and contains the fluid from the internal compartment 250 to reduce the chance of fluid exchange and directs the fluid from a first port 276 to a second port 278 via internal fluid passageways or channels.

The fluidic components 274 include a plurality of fluid pumps, such as one or more pumps 280, 282, and one or more valve 284 mounted into the fluidic manifold 208 in fluidic communication with a manifold passageway to transfer fluid from the first port 276 to the second port 278. The pump(s) 280 and the valve(s) 284 are in fluid communication with a fluid passageway (e.g., a single fluid passageway) between the ports 276, 278. The fluidic components 274 also includes one or more pressure sensors 286 operably coupled to the fluidic manifold 208 and in fluidic communication with the passageway to detect a pressure of the fluid within the fluidic manifold 208.

In some examples, the fluidic components 274 are included in a planar configuration on the fluidic manifold 208 in which the pumps 280, 282, valve 284, and pressure sensor 286 are mounted into the fluidic manifold 208 on a plane for slim profile within the second partition 254. The fluidic manifold 208 can include chambers 288 formed into the second major surface in which the chambers are fluidically coupled to the single passageway within the fluidic manifold 208. The chambers are configured to receive the pumps 280, 282, and valve 284 and one or more pressure sensors 286. In some examples, the fluidic manifold 208 can receive a piezoelectric pump. The fluidic manifold 208 can receive a component cover 290 over the fluidic components 274, which can be hermetically sealed to the second major surface.

In some examples, the electronic pump device 206 may include kink resistant tubing 292 that can extend through the header 226 and attach to the ports 276, 278 via components such as a barb 294 and O-rings. The kink resistant tubing 292 is or attached to the tube members (e.g., tube members 103, 105) to fluidically couple the electronic pump device 206 to the fluid reservoir and the inflatable member.

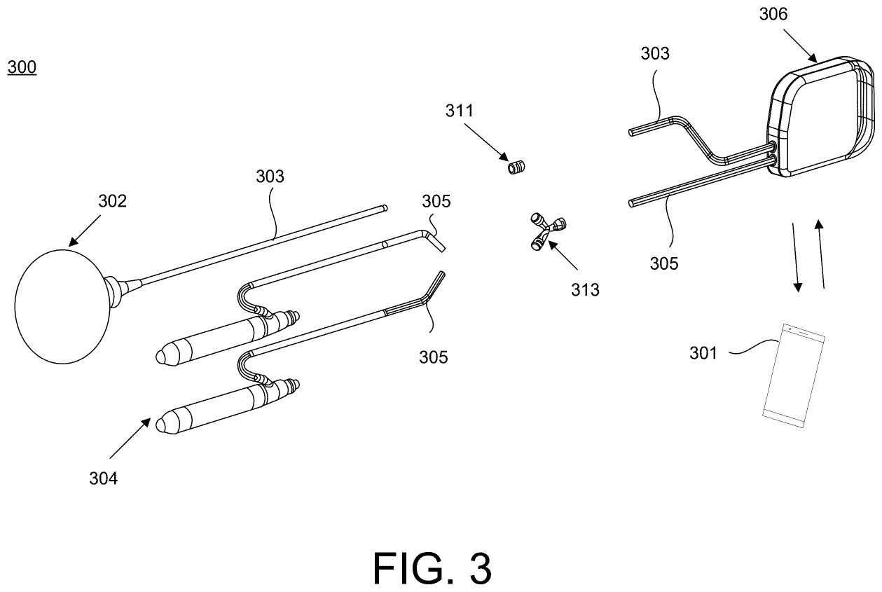

FIG. 3 illustrates a perspective of an inflatable penile prosthesis 300 according to an aspect. The inflatable penile prosthesis 300 may be an example of any of the medical devices discussed herein (e.g., including implantable medical device 100), and, therefore, may include any of the details discussed with reference to the previous figures.

The inflatable penile prosthesis 300 includes an inflatable member 304, a fluid reservoir 302, and an electronic pump device 306. The inflatable member 304 includes a pair of inflatable cylinders. The electronic pump device 306 may be an example of any of the pump devices discussed with reference to the previous figures and may include any of the details discussed herein. The electronic pump device 306 includes fluidics components such as pumps, valves, and/or sensing devices positioned in fluid passageways. The pump device 306 includes components such as, for example, one or more fluid control devices, one or more pressure sensors, and other such components. The electronic pump device 306 includes an electronic control system configured to provide for the transfer of fluid between a reservoir 302 and an inflatable member 304 via the fluidics components.

The electronic pump device 306 may include pressure sensors (e.g., pressure sensor 119-1 and/or pressure sensor 119-2). In some examples, the gauge pressure engine 108 (or a portion thereof) is included in a printed circuit board that is included in a housing of the electronic pump device 306. Fluidics components and the electronic components of the electronic pump device 306 are included in a housing. In some examples, fluidics components and electronic components in the housing define a manifold (e.g., an electronically controlled fluid manifold) that provides for the electronic control of the flow of fluid between the reservoir 302 and the inflatable member 304. In some examples, the electronic pump device 306 can communicate with an external controller 301, via respective communication modules. For example, an application stored in a memory and executed by a processor of the external controller 301 may allow the user and/or a physician to operate, view, monitor and alter operation of the inflatable penile prosthesis 300.

The inflatable penile prosthesis 300 includes one or more first tube members 303 that connect a first fluid port of the electronic pump device 306 with the reservoir 302. One or more second tube members 305 connect a second fluid port of the electronic pump device 306 with the inflatable member 304 in the form of the inflatable cylinders. In some examples, the inflatable penile prosthesis 300 includes a connector 311 that is used to connect two tube members 303 together, and a connector 313 that is used to connect two tube members 305 together.

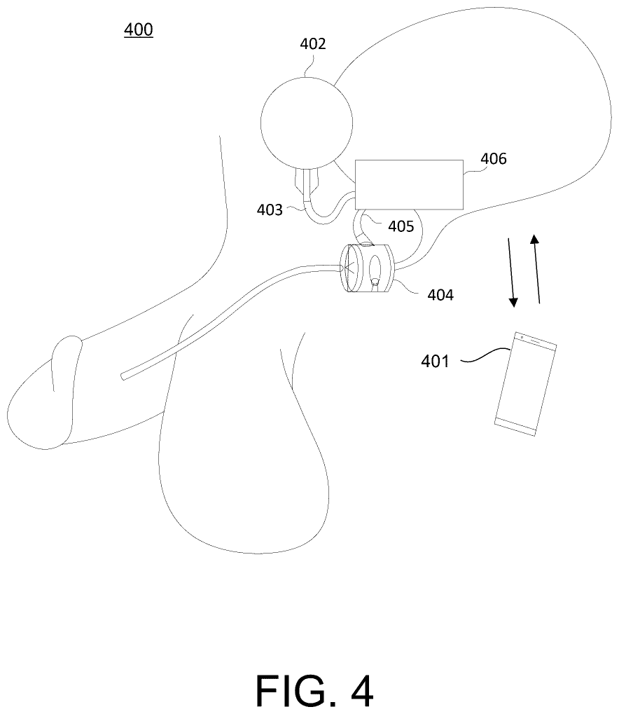

FIG. 4 illustrates a urinary control device 400 having an electronic pump device 406 according to an aspect. The urinary control device 400 may be an example of the implantable medical device 100. In some examples, the urinary control device 400 is an artificial urinary sphincter device. The electronic pump device 406 may include any of the features of the pump devices discussed herein. The urinary control device 400 includes an electronic pump device 406, a fluid reservoir 402, and a cuff 404 (e.g., an inflatable cuff).

The fluid reservoir 402 may be a pressure-regulating inflation balloon or element. The fluid reservoir 402 is in operative fluid communication with the cuff 404 via one or more tube members 403, 405. The fluid reservoir 402 is constructed of polymer material that is capable of elastic deformation to reduce fluid volume within the fluid reservoir 402 and push fluid out of the fluid reservoir 402 and into the cuff 404. However, the material of the fluid reservoir 402 can be biased or include a shape memory construct adapted to generally maintain the fluid reservoir 402 in its expanded state with a relatively constant fluid volume and pressure. In some examples, this constant level of pressure exerted from the fluid reservoir 402 to the cuff 404 will keep the cuff 404 at a desired inflated state when open fluid communication is provided between the fluid reservoir 402 and the cuff 404. In some examples, the fluid reservoir 402 is implanted into the abdominal space.

A user may use an external controller 401 to control the urinary control device 400. In some examples, the user may use the external controller 401 to inflate or deflate the cuff 404. For example, in response to the user activating an inflation cycle using the external controller 401, the external controller 401 may transmit a wireless signal to the pump device 406 to initiate the inflation cycle to transfer fluid from the fluid reservoir 402 to the cuff 404 (e.g., by opening an active valve where the pressure in the fluid reservoir 402 causes the fluid to move through the active valve to the cuff 404). In some examples, in response to the user activating a deflation cycle using the external controller 401, the external controller 401 may transmit a wireless signal to the pump device 406 to initiate the deflation cycle to transfer fluid from the cuff 404 to the fluid reservoir 402.

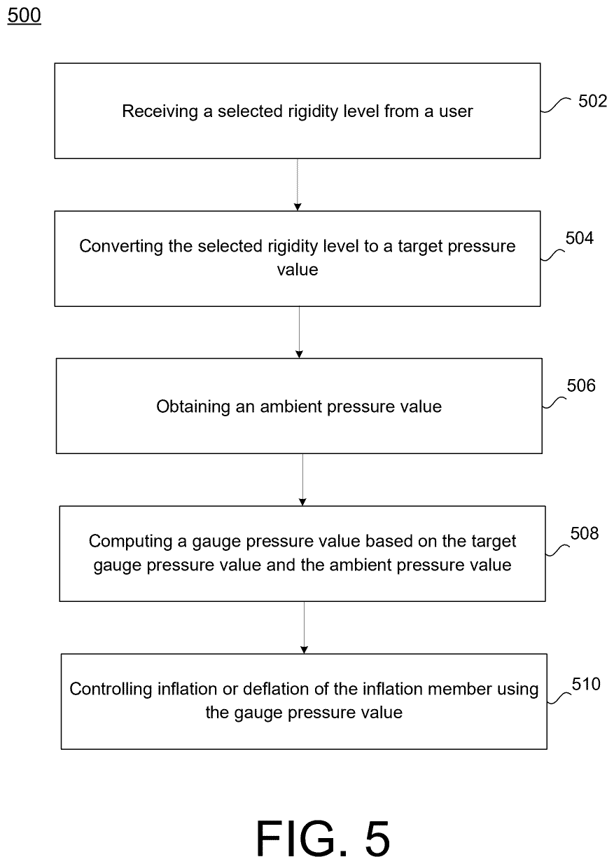

FIG. 5 illustrates a flowchart 500 depicting example operations of an electronic pump device for computing an ambient pressure value according to an aspect. Although the flowchart 500 of FIG. 5 illustrates the operations in sequential order, it will be appreciated that this is merely an example, and that additional or alternative operations may be included. Further, operations of FIG. 5 and related operations may be executed in a different order than that shown, or in a parallel or overlapping fashion.

Operation 502 includes receiving a selected rigidity level from a user. Operation 504 includes converting the selected rigidity level to a target pressure value. Operation 506 includes obtaining an ambient pressure value. Operation 508 includes computing a gauge pressure value based on the target gauge pressure value and the ambient pressure value. Operation 510 includes controlling inflation or deflation of the inflation member using the gauge pressure value.

Clause 1. An implantable medical device comprising: an electronic pump device configured to transfer fluid between a fluid reservoir and an inflatable member to inflate or deflate the inflatable member; and an external controller configured to wirelessly communicate with the electronic pump device, the external controller or the electronic pump device configured to: receive a selected rigidity level from a user; convert the selected rigidity level to a target pressure value; obtain an ambient pressure value; compute a gauge pressure value based on the target pressure value and the ambient pressure value; and control inflation or deflation of the inflatable member using the gauge pressure value.

Clause 2. The implantable medical device of clause 1, wherein the external controller or the electronic pump device is configured to: obtain a correction value; and compute the gauge pressure value based on the target pressure value, the ambient pressure value, and the correction value.

Clause 3. The implantable medical device of clause 1 or 2, wherein the external controller is configured to: display a user interface with a rigidity level control for selecting one of a plurality of rigidity levels; and receive, via the user interface, the selected rigidity level from the user.

Clause 4. The implantable medical device of clause 3, wherein the rigidity level control includes a track, and a user interface element, movable by the user, along the track, the track including a plurality of indicators representing the plurality of rigidity levels, wherein a position of the user interface element in the track indicates the selected rigidity level.

Clause 5. The implantable medical device of any one of clauses 1 to 4, wherein the external controller includes a client application executable by a user device.

Clause 6. The implantable medical device of any one of clauses 1 to 5, further comprising: a pressure sensor configured to generate pressure readings about a pressure of the inflatable member, the electronic pump device configured to control inflation of the inflatable member based on the pressure readings and the gauge pressure value.

Clause 7. The implantable medical device of clause 6, wherein the electronic pump device includes at least one pump, the electronic pump device is configured to: in response to an inflate signal, activate the at least one pump to transfer at least a portion of the fluid from the fluid reservoir to the inflatable member; and deactivate the at least one pump in response to a pressure reading, from the pressure sensor, having a pressure value that achieves the gauge pressure value.

Clause 8. The implantable medical device of clause 6, wherein the external controller or the electronic pump device is configured to compute the ambient pressure value based on the pressure readings of the pressure sensor.

Clause 9. The implantable medical device of clause 6, wherein the pressure sensor is a first pressure sensor and the pressure readings are first pressure readings, the implantable medical device further comprising: a second pressure sensor connected to the fluid reservoir, wherein the external controller or the electronic pump device is configured to compute the ambient pressure value based on second pressure readings from the second pressure sensor.

Clause 10. A method comprising: receiving a selected rigidity level from a user; converting the selected rigidity level to a target pressure value; obtaining an ambient pressure value; computing a gauge pressure value based on the target pressure value and the ambient pressure value; and controlling inflation or deflation of an inflatable member using the gauge pressure value.

Clause 11. The method of clause 10, further comprising: obtaining a correction value; and computing the gauge pressure value based on the target pressure value, the ambient pressure value, and the correction value.

Clause 12. The method of clause 10 or 11, further comprising: displaying a user interface with a rigidity level control for selecting one of a plurality of rigidity levels; and receiving, via the user interface, the selected rigidity level from the user.

Clause 13. The method of any one of clauses 10 to 12, further comprising: computing ambient pressure value based on pressure readings from a pressure sensor.

Clause 14. The method of any one of clauses 10 to 13, further comprising: in response to an inflate signal, activate at least one pump to transfer at least a portion of fluid from a fluid reservoir to the inflatable member; and deactivate the at least one pump in response to a pressure reading, from a pressure sensor, having a pressure value that achieves the gauge pressure value, the pressure sensor being connected to the inflatable member.

Clause 15. The method of clause 14, wherein the pressure sensor is a first pressure sensor and the pressure reading is a first pressure reading, the method further comprising: computing the ambient pressure value based on second pressure readings from a second pressure sensor, the second pressure sensor being connected to a fluid reservoir.

Clause 16. An implantable medical device comprising: an electronic pump device configured to transfer fluid between a fluid reservoir and an inflatable member to inflate or deflate the inflatable member; and an external controller configured to wirelessly communicate with the electronic pump device, the external controller or the electronic pump device configured to: receive a selected rigidity level from a user; convert the selected rigidity level to a target pressure value; obtain an ambient pressure value; compute a gauge pressure value based on the target pressure value and the ambient pressure value; and control inflation or deflation of the inflatable member using the gauge pressure value.

Clause 17. The implantable medical device of clause 16, wherein the external controller or the electronic pump device is configured to: obtain a correction value; and compute the gauge pressure value based on the target pressure value, the ambient pressure value, and the correction value.

Clause 18. The implantable medical device of clause 16, wherein the external controller is configured to: display a user interface with a rigidity level control for selecting one of a plurality of rigidity levels; and receive, via the user interface, the selected rigidity level from the user.

Clause 19. The implantable medical device of clause 18, wherein the rigidity level control includes a track, and a user interface element, movable by the user, along the track, the track including a plurality of indicators representing the plurality of rigidity levels, wherein a position of the user interface element in the track indicates the selected rigidity level.

Clause 20. The implantable medical device of clause 16, wherein the external controller includes a client application executable by a user device.

Clause 21. The implantable medical device of clause 16, further comprising: a pressure sensor configured to generate pressure readings about a pressure of the inflatable member, the electronic pump device configured to control inflation of the inflatable member based on the pressure readings and the gauge pressure value.

Clause 22. The implantable medical device of clause 21, wherein the electronic pump device includes at least one pump, the electronic pump device is configured to: in response to an inflate signal, activate the at least one pump to transfer at least a portion of the fluid from the fluid reservoir to the inflatable member; and deactivate the at least one pump in response to a pressure reading, from the pressure sensor, having a pressure value that achieves the gauge pressure value.

Clause 23. The implantable medical device of clause 21, wherein the external controller or the electronic pump device is configured to compute the ambient pressure value based on the pressure readings of the pressure sensor.

Clause 24. The implantable medical device of clause 21, wherein the pressure sensor is a first pressure sensor, and the pressure readings are first pressure readings, the implantable medical device further comprising: a second pressure sensor connected to the fluid reservoir, wherein the external controller or the electronic pump device is configured to compute the ambient pressure value based on second pressure readings from the second pressure sensor.

Clause 25. An implantable medical device comprising: at least one processor; and at least one non-transitory computer-readable medium storing executable instructions that when executed by the at least one processor execute operations, the operations comprising: receiving a selected rigidity level from a user; converting the selected rigidity level to a target pressure value; obtaining an ambient pressure value; computing a gauge pressure value based on the target pressure value and the ambient pressure value; and controlling inflation or deflation of an inflatable member using the gauge pressure value.

Clause 26. The implantable medical device of clause 25, wherein the operations further comprise: obtaining a correction value; and computing the gauge pressure value based on the target pressure value, the ambient pressure value, and the correction value.

Clause 27. The implantable medical device of clause 25, wherein the operations further comprise: displaying a user interface with a rigidity level control for selecting one of a plurality of rigidity levels; and receiving, via the user interface, the selected rigidity level from the user.

Clause 28. The implantable medical device of clause 27, wherein the rigidity level control includes a track, and a user interface element, movable by the user, along the track, the track including a plurality of indicators representing the plurality of rigidity levels, wherein a position of the user interface element in the track indicates the selected rigidity level.

Clause 29. The implantable medical device of clause 25, wherein the operations further comprising: generating pressure readings about a pressure of the inflatable member; and controlling inflation of the inflatable member based on the pressure readings and the gauge pressure value.

Clause 30. The implantable medical device of clause 29, wherein the operations further comprise: in response to an inflate signal, activating at least one pump to transfer at least a portion of fluid from a fluid reservoir to the inflatable member; and deactivating the at least one pump in response to a pressure reading, from a pressure sensor, having a pressure value that achieves the gauge pressure value.

Clause 31. The implantable medical device of clause 25, wherein the operations further comprise: computing ambient pressure value based on pressure readings from a pressure sensor.

Clause 32. A method comprising: receiving a selected rigidity level from a user; converting the selected rigidity level to a target pressure value; obtaining an ambient pressure value; computing a gauge pressure value based on the target pressure value and the ambient pressure value; and controlling inflation or deflation of an inflatable member using the gauge pressure value.

Clause 33. The method of clause 32, further comprising: obtaining a correction value; and computing the gauge pressure value based on the target pressure value, the ambient pressure value, and the correction value.

Clause 34. The method of clause 32, further comprising: displaying a user interface with a rigidity level control for selecting one of a plurality of rigidity levels; and receiving, via the user interface, the selected rigidity level from the user.

Clause 35. The method of clause 32, further comprising: computing ambient pressure value based on pressure readings from a pressure sensor.

Detailed embodiments are disclosed herein. However, it is understood that the disclosed embodiments are merely examples, which may be embodied in various forms. Therefore, specific structural and functional details disclosed herein are not to be interpreted as limiting, but merely as a basis for the claims and as a representative basis for teaching one skilled in the art to variously employ the embodiments in virtually any appropriately detailed structure. Further, the terms and phrases used herein are not intended to be limiting, but to provide an understandable description of the present disclosure.

The terms “a” or “an,” as used herein, are defined as one or more than one. The term “another,” as used herein, is defined as at least a second or more. The terms “including” and/or “having”, as used herein, are defined as comprising (i.e., open transition). The term “coupled” or “moveably coupled,” as used herein, is defined as connected, although not necessarily directly and mechanically.

In general, the embodiments are directed to bodily implants. The term patient or user may hereafter be used for a person who benefits from the medical device or the methods disclosed in the present disclosure. For example, the patient can be a person whose body is implanted with the medical device or the method disclosed for operating the medical device by the present disclosure. For example, in some embodiments, the patient may be a human.

While certain features of the described implementations have been illustrated as described herein, many modifications, substitutions, changes and equivalents will now occur to those skilled in the art. It is, therefore, to be understood that the appended claims are intended to cover all such modifications and changes as fall within the scope of the embodiments.

What is claimed is:

1. An implantable medical device comprising:
an electronic pump device configured to transfer fluid between a fluid reservoir and an inflatable member to inflate or deflate the inflatable member; and
an external controller configured to wirelessly communicate with the electronic pump device,
the external controller or the electronic pump device configured to:
receive a selected rigidity level from a user;
convert the selected rigidity level to a target pressure value;
obtain an ambient pressure value;
compute a gauge pressure value based on the target pressure value and the ambient pressure value; and
control inflation or deflation of the inflatable member using the gauge pressure value.

2. The implantable medical device of claim 1, wherein the external controller or the electronic pump device is configured to:
obtain a correction value; and
compute the gauge pressure value based on the target pressure value, the ambient pressure value, and the correction value.

3. The implantable medical device of claim 1, wherein the external controller is configured to:
display a user interface with a rigidity level control for selecting one of a plurality of rigidity levels; and
receive, via the user interface, the selected rigidity level from the user.

4. The implantable medical device of claim 3, wherein the rigidity level control includes a track, and a user interface element, movable by the user, along the track, the track including a plurality of indicators representing the plurality of rigidity levels, wherein a position of the user interface element in the track indicates the selected rigidity level.

5. The implantable medical device of claim 1, wherein the external controller includes a client application executable by a user device.

6. The implantable medical device of claim 1, further comprising:
a pressure sensor configured to generate pressure readings about a pressure of the inflatable member,
the electronic pump device configured to control inflation of the inflatable member based on the pressure readings and the gauge pressure value.

7. The implantable medical device of claim 6, wherein the electronic pump device includes at least one pump, the electronic pump device is configured to:
in response to an inflate signal, activate the at least one pump to transfer at least a portion of the fluid from the fluid reservoir to the inflatable member; and
deactivate the at least one pump in response to a pressure reading, from the pressure sensor, having a pressure value that achieves the gauge pressure value.

8. The implantable medical device of claim 6, wherein the external controller or the electronic pump device is configured to compute the ambient pressure value based on the pressure readings of the pressure sensor.

9. The implantable medical device of claim 6, wherein the pressure sensor is a first pressure sensor, and the pressure readings are first pressure readings, the implantable medical device further comprising:
a second pressure sensor connected to the fluid reservoir,
wherein the external controller or the electronic pump device is configured to compute the ambient pressure value based on second pressure readings from the second pressure sensor.

10. An implantable medical device comprising:
at least one processor; and
at least one non-transitory computer-readable medium storing executable instructions that when executed by the at least one processor execute operations, the operations comprising:
receiving a selected rigidity level from a user;
converting the selected rigidity level to a target pressure value;
obtaining an ambient pressure value;
computing a gauge pressure value based on the target pressure value and the ambient pressure value; and
controlling inflation or deflation of an inflatable member using the gauge pressure value.

11. The implantable medical device of claim 10, wherein the operations further comprise:
obtaining a correction value; and
computing the gauge pressure value based on the target pressure value, the ambient pressure value, and the correction value.

12. The implantable medical device of claim 10, wherein the operations further comprise:
displaying a user interface with a rigidity level control for selecting one of a plurality of rigidity levels; and
receiving, via the user interface, the selected rigidity level from the user.

13. The implantable medical device of claim 12, wherein the rigidity level control includes a track, and a user interface element, movable by the user, along the track, the track including a plurality of indicators representing the plurality of rigidity levels, wherein a position of the user interface element in the track indicates the selected rigidity level.

14. The implantable medical device of claim 10, the operations further comprising:
generating pressure readings about a pressure of the inflatable member; and
controlling inflation of the inflatable member based on the pressure readings and the gauge pressure value.

15. The implantable medical device of claim 14, wherein the operations further comprise:
in response to an inflate signal, activating at least one pump to transfer at least a portion of fluid from a fluid reservoir to the inflatable member; and
deactivating the at least one pump in response to a pressure reading, from a pressure sensor, having a pressure value that achieves the gauge pressure value.

16. The implantable medical device of claim 10, wherein the operations further comprise:
computing ambient pressure value based on pressure readings from a pressure sensor.

17. A method comprising:
receiving a selected rigidity level from a user;
converting the selected rigidity level to a target pressure value;
obtaining an ambient pressure value;
computing a gauge pressure value based on the target pressure value and the ambient pressure value; and
controlling inflation or deflation of an inflatable member using the gauge pressure value.

18. The method of claim 17, further comprising:
obtaining a correction value; and
computing the gauge pressure value based on the target pressure value, the ambient pressure value, and the correction value.

19. The method of claim 17, further comprising:
displaying a user interface with a rigidity level control for selecting one of a plurality of rigidity levels; and
receiving, via the user interface, the selected rigidity level from the user.

20. The method of claim 17, further comprising:
computing ambient pressure value based on pressure readings from a pressure sensor.
